(19) United States
(12) Patent Application Publication
(10) Pub. No.: US 2026/0118365 A1
(43) Pub. Date: Apr. 30, 2026

(54) METHODS AND SYSTEMS FOR GLYCOPROTEIN ASSAYS

(71) Applicant: Seer, Inc., Redwood City, CA (US)

(72) Inventors: Daniel HORNBURG, Redwood City, CA (US); Shadi ROSHDIFERDOSI, Redwood City, CA (US); Moaraj HASAN,, 3800 Bridge Parkway, CA (US)

(21) Appl. No.: 19/229,874

(22) Filed: Jun. 5, 2025

Related U.S. Application Data
(63) Continuation of application No. PCT/US2023/082692, filed on Dec. 6, 2023.
(60) Provisional application No. 63/386,300, filed on Dec. 6, 2022.

(51) Int. Cl. G01N 33/68

(52) U.S. Cl. CPC G01N 33/6842; G01N 33/6848; G01N 2570/00

(57) ABSTRACT

In some aspects, the present disclosure provides a method comprising: (a) incubating a composition comprising a plurality of different proteins with one or more surfaces to form a protein corona on the one or more surfaces; (b) selectively cleaving N-linked saccharides on proteins from the protein corona; and (c) assaying proteins from the protein corona to identify one or more N-linked glycoproteins in said composition. Also disclosed are systems, kits, and compositions for performing glycoprotein assays.

### CROSS-REFERENCE

This application is a continuation of International Patent Application No. PCT/US2023/082692, filed Dec. 6, 2023, which claims the benefit of U.S. Provisional Application No. 63/386,300, filed Dec. 6, 2022, which application is incorporated herein by reference in its entirety.

### BACKGROUND

Biological samples such as biofluids contain a wide variety of proteins whose presence, processing, and relative abundances may be indicative of biological state. High abundance proteins and other proteins may overshadow the signal relative to other proteins in an assay. Sample preparation, such as dilution, can further overshadow the relative signals in an assay. There is also a need for improved methods of detecting glycoproteins in these biological samples.

### SUMMARY

Provided herein is a method comprising: incubating a composition comprising a plurality of different proteins with one or more surfaces to form a protein corona on the one or more surfaces; selectively cleaving N-linked saccharides on proteins from the protein corona; and assaying proteins from the protein corona to identify one or more N-linked glycoproteins in said composition. In some embodiments, the composition has a pH between 5 and 7. In some embodiments, the composition has a pH between 9 and 10. In some embodiments, the compositions has a pH of at least about 1, 2, 3, 4, 5, 6, 7, 8, 9, 10, 11, 12, 13, or 14. In some embodiments, the composition has a pH of at most about 1, 2, 3, 4, 5, 6, 7, 8, 9, 10, 11, 12, 13, or 14. In some embodiments, the composition comprises buffer. In some embodiments, the composition comprises Tris buffer.

In some embodiments, the composition comprises at least about 1000, 100, 10, 1, 0.1, 0.01, or 0.001 nanograms of proteins. In some embodiments, the composition comprises at most about 1000, 100, 10, 1, 0.1, 0.01, or 0.001 nanograms of proteins. In some embodiments, the composition comprises a complex biological sample. In some embodiments, the complex biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the complex biological sample comprises blood, serum, or plasma.

In some embodiments, the one or more surfaces comprise a particle. In some embodiments, the particle is a porous particle. In some embodiments, the particle is a microparticle. In some embodiments, the particle is nanoparticle. In some embodiments, the particle comprises a paramagnetic material. In some embodiments, the paramagnetic material is a superparamagnetic material. In some embodiments, the paramagnetic material comprises iron oxide.

In some embodiments, said composition is incubated with a first surface and a second surface having different physicochemical properties such that different protein coronas are formed on each surface. In some embodiments, the first surface and the second surface comprise the same sign of charge. In some embodiments, the first surface and the second surface comprise the same sign of zeta potential. In some embodiments, the first surface, the second surface, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof. In some embodiments, the first surface and the second surface are incubated in separate aliquots of the composition. In some embodiments, the protein corona from the first surface and the protein corona from the second surface are assayed in (c) separately. In some embodiments, the first surface and the second surface are incubated in a single volume of the composition.

In some embodiments, the method further comprises, before (b), washing the protein corona with a wash composition. In some embodiments, the assaying comprises mass spectrometry. In some embodiments, the mass spectrometry comprises LC-MS/MS. In some embodiments, the assaying comprises affinity-based detection. In some embodiments, the assaying comprises liquid chromatography. In some embodiments, the assaying comprises protein sequencing. In some embodiments, before (b), proteins from the protein corona are removed from the one or more surfaces. In some embodiments, after (b), proteins from the protein corona are removed from the one or more surfaces. In some embodiments, proteins from the protein corona are removed from the one or more surfaces, at least in part, during (b). In some embodiments, (b) comprises incubating proteins from the protein corona, or peptide fragments thereof, with an endoglycosidase. In some embodiments, the endoglycosidase is PNGase F, PNGase A, Endoglycosidase H, Endoglycosidase F, α2-3,6,8,9 Neuraminidase A, β1-4 Galactosidase S, β-N-acetylhexosaminidase, or combinations thereof. In some embodiments, before (c), a protease is incubated with proteins from the protein corona. In some embodiments, the protease is incubated with proteins from the protein corona, at least in part, during (b). In some embodiments, the protease is incubated with proteins from the protein corona before (b). In some embodiments, the protease is trypsin, Chymotrypsin, lysc, proteinase K, elastase, AspN, GluC, or combinations thereof. In some embodiments, at least in part during (b), an endoglycosidase and a protease are incubated with the proteins from the protein corona at the same time.

In some embodiments, the method further comprises identifying a biological state associated with the composition based at least partially on the one or more N-linked glycoproteins detected in the composition. In some embodiments, selectively cleaving N-linked saccharides on proteins from the protein corona comprises hydrolyzing an amide linkage. In some embodiments, identifying one or more N-linked glycoproteins in said composition comprises detecting a protein having a deamidated residue. In some embodiments, identifying one or more N-linked glycoproteins in said composition comprises detecting a protein having an aspartic acid residue substituted for an asparagine residue. In some embodiments, the method further comprises quantifying the one or more N-linked glycoproteins. In some embodiments, N-linked glycoproteins are detected from a biological sample across a dynamic range of at least 6, 7, 8, 9, or 10 orders of magnitude. In some embodiments, N-linked oligosaccharides are cleaved from proteins from the protein corona. In some embodiments, the oligosaccharides include at least 3, 4, 5, 6, 7, 8, 9, 10, 15, 20, 25, or 50 monosaccharide units. In some embodiments, the oligosaccharides include no more than 200, 150, 100, 50, 40, 30, 20, or 15 monosaccharide units.

In some embodiments, at least 50, 100, 200, or 250 glycoproteins are detected in a biological sample. In some embodiments, the method further comprises detecting one or more non-glycosylated proteins. In some embodiments, at least 50, 100, 200, 250, 300, 400, 500, or 1000 non-glycosylated proteins are detected in a biological sample.

The present disclosure also provides a kit comprising: a protease; an endoglycosidase; a first dried composition comprising a first magnetic particle and a first stabilizer, wherein the first magnetic particle has a positive zeta potential; and a second dried composition comprising a second magnetic particle and a second stabilizer, wherein the second magnetic particle has a negative zeta potential. In some embodiments, use of the kit disclosed herein is for detecting N-linked glycoproteins in a biological sample. In some embodiments, detecting N-linked glycoproteins in the biological sample comprises performing a method provided herein.

The present disclosure also provides a composition comprising: a suspension comprising: a magnetic particle having a protein corona formed on the surface of the magnetic particle, wherein the protein corona is obtained by incubating the magnetic particle with a biological sample; and an endoglycosidase. In some embodiments, the suspension further comprises a protease. In some embodiments, the suspension further comprises trypsin. In some embodiments, the endoglycosidase is PNGase F. In some embodiments, the suspension comprises at 2, 3, 4, 5, 6, 7, 8, 9, or 10 distinct magnetic particles. In some embodiments, the magnetic particle is a nanoparticle. In some embodiments, the magnetic particle is a microparticle. In some embodiments, use of the composition disclosed herein is for detecting N-linked glycoproteins in the biological sample. In some embodiments, detecting N-linked glycoproteins in the biological sample comprises performing the method disclosed herein.

In certain aspects, the present disclosure relates to an apparatus comprising: a plurality of partitions; a plurality of reagent storages comprising an endoglycosidase and a protease, wherein the endoglycosidase and the protease are in the same or separate reagent storage; one or more surfaces; one or more transfer devices operably connected to the plurality of partitions, the plurality of reagent storages, and the plurality of substrates; and a computer comprising at least one processor and instructions executable by the at least one processor to perform steps comprising: generating, using the one or more transfer devices, a suspension in one or more of the partitions, wherein the suspension comprises at least one of the one or more surfaces and a biological sample; incubating the suspension to form a protein corona on the one or more surfaces; isolating the protein corona from the suspension; and digesting proteins from the protein corona using the endoglycosidase and protease, wherein the endoglycosidase and protease digest the proteins at the same or different times. In some embodiments, use of the apparatus disclosed herein is for detecting N-linked glycoproteins in the biological sample. In some embodiments, detecting N-linked glycoproteins in the biological sample comprises performing the method disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIG.”, “FIGS.”, “FIGURE.” or “FIGURES.” herein) of which:

FIG. 1 a high-level overview of workflows used to compare NP panel and Fe-NTA beads for glycoprotein analysis.

FIG. 2A is an example showing the number of peptides and glycopeptides identified using neat, Fe-NTA enrichment, and NP panels.

FIG. 2B is an example showing glycoforms identified using Fe-NTA enrichment and NP panel workflows.

FIG. 3A is an example showing depth of coverage for Fe-NTA enrichment and NP panel workflows.

FIG. 3B is an example showing coefficient of variance (CV) accumulation for neat, Fe-NTA enrichment, and NP panel workflows.

FIG. 3C is an example show a Venn diagrams. The left diagram shows for glycopeptides detected using the NP panel without PNGaseF treatment and with PNGaseF treatment. The right diagram shows for glycopeptides detected using Fe-NTA enrichment without PNGaseF treatment and with PNGaseF treatment.

FIG. 4 shows a computer system that is programmed or otherwise configured to implement methods provided herein.

### DETAILED DESCRIPTION

In some aspects, the present disclosure provides a method for identifying one or more N-linked glycoproteins. In some embodiments, the method comprises incubating a composition comprising a plurality of different proteins with one or more surfaces to form a protein corona on the one or more surfaces. In some embodiments, the method comprises selectively cleaving N-linked saccharides on proteins from the protein corona. In some embodiments, the method comprises assaying proteins from the protein corona to identify one or more N-linked glycoproteins in said composition.

The methods for incubating the composition are not particularly limited and may be any conditions sufficient to form a protein corona on the one or more surfaces. Methods of forming protein coronas are disclosed in U.S. Pat. No. 11,428,688, which is hereby incorporated by reference in its entirety. In some embodiments, the composition is incubated with the surface at a temperature of at least about 5, 10, 15, 20, 25, 30, 37, 40, 45, 50, 55, 60, 65, 70, 75, 80, 85, 90, or 95° C. In some embodiments, the composition is incubated with the surface at a temperate of at most about 5, 10, 15, 20, 25, 30, 37, 40, 45, 50, 55, 60, 65, 70, 75, 80, 85, 90, or 95° C. In some embodiments, the composition is incubated with the surface at a temperature of about 5° C. to 95° C., 10° C. to 90° C., 15° C. to 85° C., 20° C. to 80° C., 25° C. to 75° C., 30° C. to 70° C., 35° C. to 65° C., 40° C. to 60° C., 45° C. to 55° C., 50° C. to 95° C., or about 37° C. In some embodiments, the composition is incubated with the surface for at least about 5 minutes, 10 minutes, 15 minutes, 20 minutes, 25 minutes, 30 minutes, 35 minutes, 40 minutes, 45 minutes, 50 minutes, 55 minutes, or 1 hour. In some embodiments, the composition is incubated with the surface for at most 5 minutes, 10 minutes, 15 minutes, 20 minutes, 25 minutes, 30 minutes, 35 minutes, 40 minutes, 45 minutes, 50 minutes, 55 minutes, or 1 hour. In some embodiments, the composition is incubated with the surface for at least about 5 minutes to 1 hour, 10 minutes to 55 minutes, 15 minutes to 50 minutes, 20 minutes to 45 minutes, 25 minutes to 40 minutes, or 30 minutes to 35 minutes. In some embodiments, the composition is incubated for at least 15 minutes at a temperate of at least 20° C.

As used herein, the term “protein corona” generally refers to the plurality of different proteins that bind to a surface. The term “protein corona” generally refers to proteins and optionally other plasma components that bind to surfaces (e.g., nanoparticles) when they come into contact with biological samples or a biological system. For use herein, the term “protein corona” also encompasses both the soft and hard protein corona as referred to in Milani et al. “Reversible versus Irreversible Binding of Transferring to Polystyrene Nanoparticles: Soft and Hard Corona” ACS NANO, 2012, 6(3), pp. 2532-2541; Mirshafiee et al. “Impact of protein pre-coating on the protein corona composition and nanoparticle cellular uptake” Biomaterials vol. 75, January 2016 pp. 295-304, Mahmoudi et al. “Emerging understanding of the protein corona at the nano-bio interfaces” Nanotoday 11(6) December 2016, pp. 817-832, and Mahmoudi et al. “Protein-Nanoparticle Interactions: Opportunities and Challenges” Chem. Rev., 2011, 111(9), pp. 5610-5637, the contents of which are incorporated by reference in their entireties. As described therein, an adsorption curve may show the build-up of a strongly bound monolayer up to the point of monolayer saturation (at a geometrically defined protein-to-NP ratio), beyond which a secondary, weakly bound layer is formed. While the first layer is irreversibly bound (hard corona), the secondary layer (soft corona) may exhibit dynamic exchange. Proteins that adsorb with high affinity may form the “hard” corona, comprising tightly bound proteins that do not readily desorb, and proteins that adsorb with low affinity may form the “soft” corona, comprising loosely bound proteins. Soft and hard corona can also be characterized based on their exchange times. Hard corona may show much larger exchange times in the order of several hours. See, e.g., M. Rahman et al. Protein-Nanoparticle Interactions, Spring Series in Biophysics 15, 2013, incorporated by reference in its entirety.

In some embodiments, an N-linked saccharide on proteins from the protein corona may be selectively cleaved using a suitable enzyme. In some embodiments, the enzyme is an endoglycosidase, such as PNGase F (peptide:N-glycosidase F). The N-linked saccharide may be selectively cleaved from proteins from the protein corona by appropriate cleavage chemistry applied to the protein while still intact, or alternatively by applying appropriate chemistry applied to peptide fragments derived from the proteins. In either case, an N-linked saccharide on proteins from the protein corona can be selectively cleaved. In some cases, the N-linked saccharides are oligosaccharides. The oligosaccharides may include, for example, 3 to 100 monosaccharide units, 3 to 50 monosaccharide units, or 3 to 20 monosaccharide units. In some cases, the oligosaccharide may have a structure that is a suitable substrate for enzymatic cleavage. For example, the oligosaccharide may be a substrate for cleavage by PNGase F, or other endoglycosidases.

The N-linked saccharides may be selectively cleaved before, during, or after removing proteins in the protein corona from the one or more surfaces. In some embodiments, the N-linked saccharides are selectively cleaved before removing proteins in the protein corona from the one or more surfaces. For example, the protein corona on the one or more surfaces may be incubated with a suitable enzyme to cleave N-linked saccharides. In some embodiments, the N-linked saccharides are selectively cleaved, at least part, at the same time as removing proteins in the protein corona from the one or more surfaces. For example, the protein corona on the one or more surfaces may be incubated with both a suitable enzyme to cleave N-linked saccharides and a protease. The protease cleavage may act to remove the proteins from the one or more surfaces by producing peptide fragments, and at the same time the suitable enzyme can cleave the N-linked saccharides in the proteins (or peptide derivatives). In some embodiments, the N-linked saccharides in the proteins are selectively cleaved after removing proteins in the protein corona from the one or more surfaces. For example, the protein corona on the one or more surfaces may be first incubated with a protease to remove the proteins from the one or more surfaces by fragmenting the proteins into peptides, and then the peptides can be incubated with a suitable enzyme to cleave N-linked saccharides. The proteins may be optionally separated from the one or more surfaces after incubation with the protease, but before incubated with the suitable enzyme to cleave N-linked saccharides.

In some embodiments, the proteins may be further modified before, during, or after selectively cleaving N-linked saccharides from proteins in the protein corona. The modification may be selected based on the particular application and assay techniques application. For example, proteins may be prepared for mass spectrometry analysis by performing alkylation.

The N-linked glycoproteins from the protein corona may be identified using various techniques. In some embodiments, affinity-based techniques, such as ELISA, can be used to detect N-linked proteins. For example, antibodies that bind an epitope containing the cleaved residue may be used. In some embodiments, mass spectrometric analysis (e.g., MS, LC-MS, LC-MS/MS) may be performed. For example, proteins or their peptide fragments may be detected, and the N-linked glycoproteins identified by changes atomic weight that indicate a cleavage site. This could be, for example, identifying a peptide having an aspartic acid residue substituted for an asparagine residue, which would indicate cleavage by an endoglycosidase, such as PNGase F. In some embodiments, protein sequencing methods may be used. For example, sequencing methods available from Nautilus Biosciences, Quantum Si, and others. An N-linked glycoprotein may be identified by, for example, sequencing a peptide having an aspartic acid residue substituted for an asparagine residue, which would indicate cleavage by an endoglycosidase, such as PNGase F.

In some cases, the methods are advantageous because they allow large numbers of proteins and glycoproteins to be identified in one assay. For example, the methods may identify at least 100 glycoproteins and at least 700 proteins. In some cases, the methods are advantageous because they introduce less bias compared known glycoprotein enrichment methods. For example, Fe-NTA enrichment may favor particular glycosylation structures, whereas the methods disclosed herein may not. In some cases, the methods are advantageous because the allow analysis of glycoproteins across a larger dynamic range. For example, lower abundance glycoproteins may be identified in biological samples that cannot be identified using other techniques.

In some aspects, the present disclosure provides a kit for performing the methods disclosed herein. In some embodiments, the kit includes a protease. In some embodiments, the kit includes an endoglycosidase. In some embodiments, the kit includes a first dried composition comprising a first magnetic particle and a first stabilizer, wherein the first magnetic particle has a positive zeta potential. a first dried composition comprising a first magnetic particle and a first stabilizer, wherein the first magnetic particle has a positive zeta potential a second dried composition comprising a second magnetic particle and a second stabilizer, wherein the second magnetic particle has a negative zeta potential. In some embodiments, the first stabilizer and the second stabilizer are the same. In some embodiments, the first stabilizer and the second stabilizer are both a saccharide. In some embodiments, the first dried composition, the second dried composition, or both, are lyophilized.

In some aspects, the present disclosure provides a composition for use in the methods disclosed herein. In some embodiments, the composition includes a suspension. In some embodiments, the suspension comprises a magnetic particle having a protein corona formed on the surface of the magnetic particle. In some embodiments, the protein corona is obtained by incubating the magnetic particle with a biological sample. In some embodiments, the suspension comprises an endoglycosidase.

In some aspects, the present disclosure provides an apparatus for use with the methods disclosed herein. In some embodiments, the apparatus comprises a plurality of partitions. In some embodiments, the apparatus comprises a plurality of reagent storages having an endoglycosidase and a protease, wherein the endoglycosidase and the protease are in the same or separate reagent storage. In some embodiments, the apparatus comprises one or more surfaces (e.g., magnetic particles). In some embodiments, the apparatus comprises one or more transfer devices operably connected to the plurality of partitions, the plurality of reagent storages, and the plurality of substrates. In some embodiments, the apparatus comprises a computer comprising at least one processor and instructions executable by the at least one processor to perform steps. In some embodiments, the steps include generating, using the one or more transfer devices, a suspension in one or more of the partitions, wherein the suspension comprises at least one of the surfaces and a biological sample. In some embodiments, the steps include incubating the suspension to form a protein corona on the one or more surfaces. In some embodiments, the steps include digesting proteins from the protein corona using the endoglycosidase and protease, wherein the endoglycosidase and protease digest the proteins at the same or different times.

The apparatus may, in some embodiments, include various other components for performing analysis of protein coronas on surfaces. Examples of such components and other features that may be included in the apparatus are disclosed in U.S. Publication No. 2021/0285958, which is hereby incorporated by reference in its entirety.

### Biological Sample

The present disclosure provides systems, compositions, kits and methods for assaying a biological sample for glycoproteins. In some cases, a biological sample may comprise a cell or be cell-free. In some cases, a biological sample may comprise a biofluid, such as blood, serum, plasma, urine, or cerebrospinal fluid (CSF). In some cases, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract. In some cases, a biological sample may be obtained from a subject. In some cases, the subject may be a human or a non-human. In some cases, the subject may be a plant, a fungus, or an archaeon. In some cases, a biological sample can contain a plurality of proteins or proteomic data, which may be analyzed after adsorption or binding of proteins to the one or more surfaces of the various sensor element (e.g., particle) types in a panel and subsequent digestion of protein coronas.

In some cases, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, a biological sample may comprise multiple biological samples (e.g., pooled plasma from multiple subjects, or multiple tissue samples from a single subject). In some cases, a biological sample may comprise a single type of biofluid or biomaterial from a single source.

In some cases, a biological sample may be diluted or pre-treated. In some cases, a biological sample may undergo depletion (e.g., the biological sample comprises serum) prior to or following contact with a surface disclosed herein. In some cases, a biological sample may undergo physical (e.g., homogenization or sonication) or chemical treatment prior to or following contact with a surface disclosed herein. In some cases, a biological sample may be diluted prior to or following contact with a surface disclosed herein. In some cases, a dilution medium may comprise buffer or salts, or be purified water (e.g., distilled water). In some cases, a biological sample may be provided in a plurality partitions, wherein each partition may undergo different degrees of dilution. In some cases, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. In some cases, a biological sample may comprise may undergo at most about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. In some cases, a biological sample may comprise may undergo about 1.1-fold to 1000-fold, 1.2-fold to 500-fold, 1.3-fold to 200-fold, 1.4-fold to 100-fold, 1.5-fold to 75-fold, 2-fold to 50-fold, 3-fold to 40-fold, 4-fold to 30-fold, 5-fold to 20-fold, 6-fold to 15-fold, 8-fold to 12-fold, or 10-fold to1000-fold dilution. In some cases, the biological sample may be diluted using a buffer that modified pH of the biological sample. In some case, the pH may be modified to at least about 2, 3, 4, 5, 6, 7, 8, 9, 10. In some case, the pH may be modified to at most about 2, 3, 4, 5, 6, 7, 8, 9, 10. In some case, the pH may be modified to about 2 to 10, 3 to 9, 4 to 8, 5 to 7, 6 to 10. In some case, the pH may be modified to 9-10. In some cases, the pH is modified by dilution with a pH 9.5 Tris buffer. In some cases, the biological sample be separated into portions, and the portions may be adjusted to different pHs before processing using the methods disclosed herein.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

### Proteomic Analysis

As used herein, “proteomic analysis”, “protein analysis”, and the like, may refer to any system or method for analyzing proteins, including glycoproteins, in a sample, including the systems and methods disclosed herein. The present disclosure systems and methods for assaying using one or more surfaces. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least about 5, 6, 7, 8, 9, 10, 15, or 20 magnitudes. In some cases, a panel including any number of distinct particle types disclosed herein, may enrich and identify biomolecules over a dynamic range of at most about 5, 6, 7, 8, 9, 10, 15, or 20 magnitudes. In some cases, a panel including any number of distinct particle types disclosed herein, may enrich and identify biomolecules over a dynamic range of about 5 to 20, 6 to 15, 7 to 10, or 8 to 9 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a particle coated with an outer layer of silica and a particle coated with an outer layer of poly(dimethyl aminopropyl methacrylamide) (PDMAPMA). A particle panel consistent with the present disclosure could also comprise two or more particles selected from the group consisting of silica coated particle, an N-(3-Trimethoxysilylpropyl) diethylenetriamine coated particle, a PDMAPMA coated particle, a carboxyl-functionalized polyacrylic acid coated particle, an amino surface functionalized particle, a polystyrene carboxyl functionalized particle, and a dextran coated particle. A particle panel consistent with the present disclosure may also comprise two or more particles selected from the group consisting of a surfactant free carboxylate functionalized particle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-saccharide functionalized polystyrene particles, amine functionalized polystyrene particles, polystyrene carboxyl functionalized particles, ubiquitin functionalized polystyrene particles, dextran coated particles, or any combination thereof. In some cases, the particle panel may comprise silica-coated particles, amine-functionalized particles, amine-functionalized polymer-coated particles, and carboxylate-functionalized particles.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group, a carbamoyl group, a hydroxyl group, an aldehyde group and the like. In some cases, a polar functional group may comprise a primary amine group, a secondary amine group, a tertiary amine group, a quaternary amine group, a cyclic secondary amine group, a primary amide group, a secondary amide group, a tertiary amide group, an imine group, a pyridyl group, a pyrimidine group, a pyrrolidinium group, an imidazole group, a guanidine group, a guanidinium group, or any combination thereof.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle. In some cases, a positively charged particle may have a zeta potential of more than about 0, 5, 10, 15, 20, 25, 50, or 100 mV. In some cases, a negative charged particle may have a zeta potential of less than about 0, −5, −10, −15, −20, −25, −50, or −100 mV. In some cases, a positively charged particle may have a zeta potential of more than about 0 to 100, 5 to 50, 10 to 25, or 15 to 20 mV.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2,3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a surface functionalization may comprise pKa of about 1 to 14, 2 to 13, 3 to 12, 4 to 11, 5 to 10, 6 to 9, or 7 to 8. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. In some cases, a linker may be about 0.5 to 30, 1 to 25, 2 to 20, 3 to 15, 4 to 9, 5 to 8, or 6 to 7 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique glycoproteins in a biological sample. In some cases, a method may identify at least about 1, 2, 3, 4, 5, 6, 7, 8, 9, 10, 20, 30, 40, 50, 60, 70, 80, 90, 100, or 200 unique glycoproteins. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique glycoproteins. In some cases, a method may identify about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, or 100 to 10000 unique glycoproteins.

In some cases, a method of the present disclosure may identify a large number of unique biomolecules (e.g., proteins) in a biological sample (e.g., a biofluid). In some cases, a surface disclosed herein may be incubated with a biological sample to adsorb at least about 1000, 2000, 3000, 4000, 5000, 6000, 7000, 8000, 9000, or 10000 unique biomolecules. In some cases, a surface disclosed herein may be incubated with a biological sample to adsorb at most about 1000, 2000, 3000, 4000, 5000, 6000, 7000, 8000, 9000, or 10000 unique biomolecules. In some cases, a surface disclosed herein may be incubated with a biological sample to adsorb at least about 1000 to 10000, 2000 to 9000, 3000 to 8000, 4000 to 7000, or 5000 to 6000 unique biomolecules. In some cases, a surface disclosed herein may be incubated with a biological sample to adsorb at least about 1000, 2000, 3000, 4000, 5000, 6000, 7000, 8000, 9000, or 10000 unique biomolecule groups. In some cases, a surface disclosed herein may be incubated with a biological sample to adsorb at most about 1000, 2000, 3000, 4000, 5000, 6000, 7000, 8000, 9000, or 10000 unique biomolecule groups. In some cases, a surface disclosed herein may be incubated with a biological sample to adsorb about 1000 to 10000, 2000 to 9000, 3000 to 8000, 4000 to 7000, or 5000 to 6000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, or 100 to 10000 unique proteoforms. In some cases, a surface disclosed herein may be incubated with a biological sample to adsorb at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, or 100 to 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at most about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify about 100 to 100000, 200 to 90000, 300 to 80000, 400 to 70000, 500 to 60000, 600 to 50000, 700 to 40000, 800 to 30000, 900 to 20000, 1000 to 10000, 2000 to 9000, 3000 to 8000, 4000 to 7000, or 5000 to 6000 unique proteins. In some cases, the panels disclosed herein can be used to identify at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at most about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify about 100 to 100000, 200 to 90000, 300 to 80000, 400 to 70000, 500 to 60000, 600 to 50000, 700 to 40000, 800 to 30000, 900 to 20000, 1000 to 10000, 2000 to 9000, 3000 to 8000, 4000 to 7000, or 5000 to 6000 protein groups. In some cases, the panels disclosed herein can be used to identify at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify at most about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify about 100 to 100000, 200 to 90000, 300 to 80000, 400 to 70000, 500 to 60000, 600 to 50000, 700 to 40000, 800 to 30000, 900 to 20000, 1000 to 10000, 2000 to 9000, 3000 to 8000, 4000 to 7000, or 5000 to 6000 peptides.

In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least about 2 proteins, 5 proteins, 10 proteins, 20 proteins, 40 proteins, 60 proteins, 80 proteins, 100 proteins, 150 proteins, 200 proteins, or more. In some cases, a protein class may contain at most about 2 proteins, 5 proteins, 10 proteins, 20 proteins, 40 proteins, 60 proteins, 80 proteins, 100 proteins, 150 proteins, 200 proteins, or more. In some cases, a protein class may contain about 2 proteins to 200 proteins, 5 proteins to 150 proteins, 10 proteins to 100 proteins, 20 proteins to 80 proteins, 40 proteins to 60 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of about 8 to 15 amino acids. In some cases, the digestion may generate an average peptide fragment length of about 12 to 18 amino acids. In some cases, the digestion may generate an average peptide fragment length of about 15 to 25 amino acids. In some cases, the digestion may generate an average peptide fragment length of about 20 to 30 amino acids. In some cases, the digestion may generate an average peptide fragment length of about 30 to 50 amino acids. In some cases, the digestion may generate an average peptide fragment length of at least about 8, 10, 15, 20, 25, 30, 35, 40, 45, 50 amino acids. In some cases, the digestion may generate an average peptide fragment length of at most about 8, 10, 15, 20, 25, 30, 35, 40, 45, 50 amino acids.

In some cases, proteins or peptides may be prepared for mass spectrometry. In some cases, proteins or peptides may be treated with an alkylating agent. For example, proteins or peptides may be treated with N-ethylmaleimide and iodoacetamide. In some case, proteins or peptides may be treated with a reducing agent. For example, proteins or peptides are treated with dithiothreitol (DTT) or tris(2-carboxyethyl)phosphine (TCEP). In some cases, proteins or peptides are digested, alkylated, and reduced before analysis. In some cases, proteins or peptides are digested, alkylated, and reduced before analysis using mass spectrometry.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 um in diameter, a silica amino functionalized microparticle of about 0.1-0.39 um in diameter, a Jeffamine surface particle of about 0.1-0.39 um in diameter, a polystyrene microparticle of about 2.0-2.9 um in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some cases, a particle may comprise a nanoparticle. In some cases, a particle may comprise a microparticle. In some cases, a first particle, a second particle, or both particles in a particle panel are nanoparticles. In some cases, a first particle, a second particle, or both particles in a particle panel are microparticles. In some cases, a first particle may be a nanoparticle and a second particle may be a microparticle.

In some cases, a particle may comprise a diameter of at least about 10, 20, 30, 40, 50, 60, 70, 80, 90, 100, 200, 300, 400, 500, 600, 700, 800, 900, or 1000 nm. In some cases, a particle may comprise a diameter of at most about 10, 20, 30, 40, 50, 60, 70, 80, 90, 100, 200, 300, 400, 500, 600, 700, 800, 900, or 1000 nm. In some cases, a particle may comprise a diameter of about 10 to 1000, 20 to 900, 30 to 800, 40 to 700, 50 to 600, 60 to 500, 70 to 400, 80 to 300, 90 to 200, or 100 to 1000 nm. In some cases, a particle may comprise a diameter of at least about 10, 20, 30, 40, 50, 60, 70, 80, 90, 100, 200, 300, 400, 500, 600, 700, 800, 900, or 1000 μm. In some cases, a particle may comprise a diameter of at most about 10, 20, 30, 40, 50, 60, 70, 80, 90, 100, 200, 300, 400, 500, 600, 700, 800, 900, or 1000 μm. In some cases, a particle may comprise a diameter of about 10 to 1000, 20 to 900, 30 to 800, 40 to 700, 50 to 600, 60 to 500, 70 to 400, 80 to 300, 90 to 200, or 100 to 1000 μm.

In some cases, a first size of a first particle is at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1000 times a second size of a second particle. In some cases, a first size of a first particle is at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1000 times a second size of a second particle. In some cases, a first size of a first particle is about 1.1 to 1000, 1.2 to 900, 1.3 to 800, 1.4 to 700, 1.5 to 600, 1.6 to 500, 1.7 to 400, 1.8 to 300, 1.9 to 200, 2 to 100, 3 to 90, 4 to 80, 5 to 70, 10 to 60, 20 to 50, or 30 to 40 times a second size of a second particle. In some cases, a size of a first particle is within ±40% of a size of a second particle, a size of a first particle is within ±30% of a size of a second particle, a size of a first particle is within ±25% of a size of a second particle, a size of a first particle is within ±20% of a size of a second particle, a size of a first particle is within ±15% of a size of a second particle, or a size of a first particle is within ±10% of a size of a second particle. In some cases, a size of a first particle is within at least about ±10%, ±15%, ±20%, +25%, ±30%, +35%, ±40% of a size of a second particle. In some cases, a size of a first particle is within at most about ±10%, ±15%, ±20%, ±25%, ±30%, ±35%, ±40% of a size of a second particle. In some cases, a size of a first particle is within about ±10% to ±40%, ±15% to ±35%, ±20% to ±30%, ±25% to ±40% of a size of a second particle. In some cases, the first size is a first diameter, and the second size is a second diameter. In some cases, the first size is a first average size, and the second size is a second average size. In some cases, the first average size and the second average size are mean sizes or median sizes.

In some cases, a particle panel comprises at least about 1, 2, 3, 4, 5, 6, 7, 8, 9, or 10 parts of a first particle to about 1 part of a second particle. In some cases, a particle panel comprises at most about 1, 2, 3, 4, 5, 6, 7, 8, 9, or 10 parts of a first particle to about 1 part of a second particle. In some cases, a particle panel comprises about 1 to 10, 2 to 9, 3 to 8, 4 to 7, 5 to 6 parts of a first particle to about 1 part of a second particle. In some cases, a particle panel comprises about 15 parts of a first particle to about 6 parts of a second particle. In some cases, the parts are parts by weight, parts by volume, or parts by surface area. In some cases, the parts are parts by weight.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least about 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm2. In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 fg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 pg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 ng/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 μg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, 100 to 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration. In some cases, proteins in a biological sample may comprise about 1 to 30, 2 to 25, 3 to 20, 4 to 15, 5 to 10, 6 to 9, or 7 to 8 orders of magnitudes in concentration.

### Computer Systems

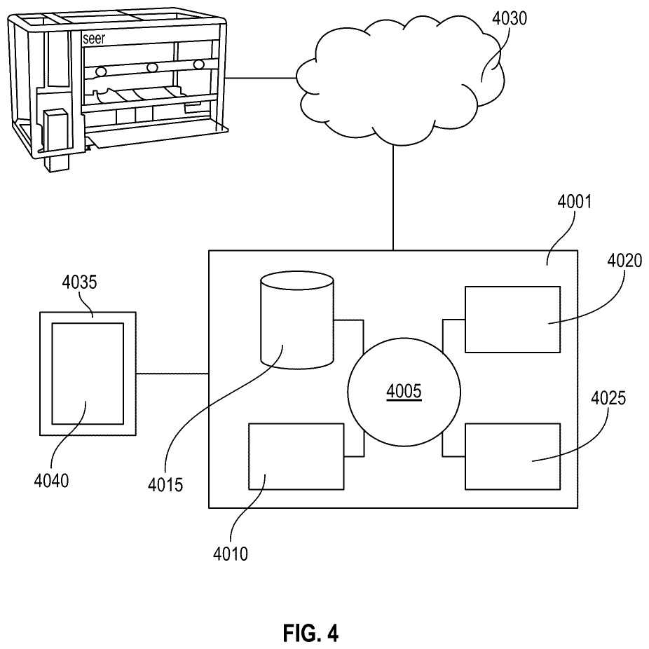

The present disclosure provides computer systems that are programmed to implement methods of the disclosure. FIG. 4 shows a computer system 4001 that is programmed or otherwise configured to, for example, contact one or more biological samples with one or more particles to form one or more biomolecule coronas and analyze the one or more biomolecule coronas with a proteomic method, genomic method, or both.

The computer system 4001 may regulate various aspects of analysis, calculation, and generation of the present disclosure, such as, for example, contacting one or more biological samples with one or more particles to form one or more biomolecule coronas and analyzing the one or more biomolecule coronas with a proteomic method, genomic method, or both. The computer system 4001 may be an electronic device of a user or a computer system that is remotely located with respect to the electronic device. The electronic device may be a mobile electronic device. The electronic device may comprise a wireless keyboard and a mouse. The electronic device may comprise a display mount (e.g., Hamilton arm).

The computer system 4001 includes a central processing unit (CPU, also “processor” and “computer processor” herein) 4005, which may be a single core or multi core processor, or a plurality of processors for parallel processing. The computer system 4001 also includes memory or memory location 4010 (e.g., random-access memory, read-only memory, flash memory), electronic storage unit 4015 (e.g., hard disk), communication interface 4020 (e.g., network adapter) for communicating with one or more other systems, and peripheral devices 4025, such as cache, other memory, data storage and/or electronic display adapters. The memory 4010, storage unit 4015, interface 4020 and peripheral devices 4025 are in communication with the CPU 4005 through a communication bus (solid lines), such as a motherboard. The storage unit 4015 may be a data storage unit (or data repository) for storing data. The computer system 4001 may be operatively coupled to a computer network (“network”) 4030 with the aid of the communication interface 4020. The network 4030 may be the Internet, an internet and/or extranet, or an intranet and/or extranet that is in communication with the Internet.

The network 4030 in some cases is a telecommunication and/or data network. The network 4030 may include one or more computer servers, which may enable distributed computing, such as cloud computing. For example, one or more computer servers may enable cloud computing over the network 4030 (“the cloud”) to perform various aspects of analysis, calculation, and generation of the present disclosure, such as, for example, contacting one or more biological samples with one or more particles to form one or more biomolecule coronas and analyzing the one or more biomolecule coronas with a proteomic method, genomic method, or both. Such cloud computing may be provided by cloud computing platforms such as, for example, Amazon Web Services (AWS), Microsoft Azure, Google Cloud Platform, and IBM cloud. The network 4030, in some cases with the aid of the computer system 4001, may implement a peer-to-peer network, which may enable devices coupled to the computer system 6001 to behave as a client or a server.

The CPU 4005 may comprise one or more computer processors and/or one or more graphics processing units (GPUs). The CPU 4005 may execute a sequence of machine-readable instructions, which may be embodied in a program or software. The instructions may be stored in a memory location, such as the memory 4010. The instructions may be directed to the CPU 4005, which may subsequently program or otherwise configure the CPU 4005 to implement methods of the present disclosure. Examples of operations performed by the CPU 4005 may include fetch, decode, execute, and writeback.

The CPU 4005 may be part of a circuit, such as an integrated circuit. One or more other components of the system 4001 may be included in the circuit. In some cases, the circuit is an application specific integrated circuit (ASIC).

The storage unit 4015 may store files, such as drivers, libraries and saved programs. The storage unit 4015 may store user data, e.g., user preferences and user programs. The computer system 4001 in some cases may include one or more additional data storage units that are external to the computer system 4001, such as located on a remote server that is in communication with the computer system 4001 through an intranet or the Internet.

The computer system 4001 may communicate with one or more remote computer systems through the network 4030. For instance, the computer system 4001 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user may access the computer system 4001 via the network 4030.

Methods as described herein may be implemented by way of machine (e.g., computer processor) executable code stored on an electronic storage location of the computer system 4001, such as, for example, on the memory 4010 or electronic storage unit 4015. The machine executable or machine-readable code may be provided in the form of software. During use, the code may be executed by the processor 4005. In some cases, the code may be retrieved from the storage unit 4015 and stored on the memory 4010 for ready access by the processor 4005. In some situations, the electronic storage unit 4015 may be precluded, and machine-executable instructions are stored on memory 4010.

The code may be pre-compiled and configured for use with a machine having a processer adapted to execute the code or may be compiled during runtime. The code may be supplied in a programming language that may be selected to enable the code to execute in a pre-compiled or as-compiled fashion.

Aspects of the systems and methods provided herein, such as the computer system 6001, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 4001 may include or be in communication with an electronic display 4035 that comprises a user interface (UI) 4040 for providing, for example, contacting one or more biological samples with one or more particles to form one or more biomolecule coronas and analyzing the one or more biomolecule coronas with a proteomic method, genomic method, or both. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 6005. The algorithm can, for example, contacting one or more biological samples with one or more particles to form one or more biomolecule coronas and analyzing the one or more biomolecule coronas with a proteomic method, genomic method, or both.

### Embodiments

In one aspect, provided herein is a method comprising: (a) incubating a composition comprising a plurality of different proteins with one or more surfaces to form a protein corona on the one or more surfaces; (b) selectively cleaving N-linked saccharides on proteins from the protein corona; and (c) assaying proteins from the protein corona to identify one or more N-linked glycoproteins in said composition.

In some embodiments, the composition has a pH between about 5 and 7.

In some embodiments, the composition has a pH between about 9 and 10.

In some embodiments, the composition has a pH of at least about 1, 2, 3, 4, 5, 6, 7, 8, 9, 10, 11, 12, 13, or 14.

In some embodiments, the composition has a pH of at most about 1, 2, 3, 4, 5, 6, 7, 8, 9, 10, 11, 12, 13, or 14.

In some embodiments, the composition comprises buffer.

In some embodiments, the composition comprises Tris buffer.

In some embodiments, the composition comprises at least about 1000, 100, 10, 1, 0.1, 0.01, or 0.001 nanograms of proteins.

In some embodiments, the composition comprises at most about 1000, 100, 10, 1, 0.1, 0.01, or 0.001 nanograms of proteins.

In some embodiments, the composition comprises a complex biological sample.

In some embodiments, the complex biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the complex biological sample comprises blood, serum, or plasma.

In some embodiments, the one or more surfaces comprise a particle.

In some embodiments, the particle is a porous particle.

In some embodiments, the particle is a microparticle.

In some embodiments, the particle is nanoparticle.

In some embodiments, the particle comprises a paramagnetic material.

In some embodiments, the paramagnetic material is a superparamagnetic material.

In some embodiments, the paramagnetic material comprises iron oxide.

In some embodiments, the composition is incubated with a first surface and a second surface having different physicochemical properties such that different protein coronas are formed on each surface.

In some embodiments, the first surface and the second surface comprise the same sign of charge.

In some embodiments, the first surface and the second surface comprise the same sign of zeta potential.

In some embodiments, the first surface, the second surface, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

In some embodiments, the first surface and the second surface are incubated in separate aliquots of the composition.

In some embodiments, the protein corona from the first surface and the protein corona from the second surface are assayed in (c) of the method provided herein separately.

In some embodiments, the first surface and the second surface are incubated in a single volume of the composition.

In some embodiments, comprising, before (b) of the method provided herein, washing the protein corona with a wash composition.

In some embodiments, the assaying comprises mass spectrometry.

In some embodiments, the mass spectrometry comprises LC-MS/MS.

In some embodiments, the assaying comprises affinity-based detection.

In some embodiments, the assaying comprises liquid chromatography.

In some embodiments, the assaying comprises protein sequencing.

In some embodiments, before (b) of the method provided herein, proteins from the protein corona are removed from the one or more surfaces.

In some embodiments, after (b) of the method provided herein, proteins from the protein corona are removed from the one or more surfaces.

In some embodiments, proteins from the protein corona are removed from the one or more surfaces, at least in part, during (b) of the method provided herein.

In some embodiments, (b) of the method provided herein comprises incubating proteins from the protein corona, or peptide fragments thereof, with an endoglycosidase.

In some embodiments, the endoglycosidase is PNGase F, PNGase A, Endoglycosidase H, Endoglycosidase F, α2-3,6,8,9 Neuraminidase A, β1-4 Galactosidase S, β-N-acetylhexosaminidase, or combinations thereof.

In some embodiments, before (c) of the method provided herein, a protease is incubated with proteins from the protein corona.

In some embodiments, the protease is incubated with proteins from the protein corona, at least in part, during (b) of the method provided herein.

In some embodiments, the protease is incubated with proteins from the protein corona before (b) of the method provided herein.

In some embodiments, the protease is trypsin, Chymotrypsin, lysc, proteinase K, elastase, AspN, GluC, or combinations thereof.

In some embodiments, at least in part during (b) of the method provided herein, an endoglycosidase and a protease are incubated with the proteins from the protein corona at the same time.

In some embodiments, the method provided herein further comprises identifying a biological state associated with the composition based at least partially on the one or more N-linked glycoproteins detected in the composition.

In some embodiments, selectively cleaving N-linked saccharides on proteins from the protein corona comprises hydrolyzing an amide linkage.

In some embodiments, identifying one or more N-linked glycoproteins in a composition provided herein comprises detecting a protein having a deamidated residue.

In some embodiments, identifying one or more N-linked glycoproteins in a composition provided herein comprises detecting a protein having an aspartic acid residue substituted for an asparagine residue.

In some embodiments, the method provided herein further comprises quantifying the one or more N-linked glycoproteins.

In some embodiments, N-linked glycoproteins are detected from a biological sample across a dynamic range of at least 6, 7, 8, 9, or 10 orders of magnitude.

In some embodiments, N-linked oligosaccharides are cleaved from proteins from the protein corona.

In some embodiments, the oligosaccharides include at least 3, 4, 5, 6, 7, 8, 9, 10, 15, 20, 25, or 50 monosaccharide units.

In some embodiments, the oligosaccharides include no more than 200, 150, 100, 50, 40, 30, 20, or 15 monosaccharide units.

In some embodiments, at least 50, 100, 200, or 250 glycoproteins are detected in a biological sample.

In some embodiments, the method provided herein further comprises detecting one or more non-glycosylated proteins.

In some embodiments, at least 50, 100, 200, 250, 300, 400, 500, or 1000 non-glycosylated proteins are detected in a biological sample.

In another aspect, provided herein is a kit comprising: (a) a protease; (b) an endoglycosidase; (c) a first dried composition comprising a first magnetic particle and a first stabilizer, wherein the first magnetic particle has a positive zeta potential; and (d) a second dried composition comprising a second magnetic particle and a second stabilizer, wherein the second magnetic particle has a negative zeta potential.

In some embodiments, the kit provided herein is used for detecting N-linked glycoproteins in a biological sample.

In some embodiments, detecting N-linked glycoproteins in the biological sample comprises performing a method provided herein.

In another aspect, provided herein is a composition comprising: a suspension comprising: i. a magnetic particle having a protein corona formed on the surface of the magnetic particle, wherein the protein corona is obtained by incubating the magnetic particle with a biological sample; and ii. an endoglycosidase.

In some embodiments, the suspension further comprises a protease.

In some embodiments, the suspension further comprises trypsin.

In some embodiments, the endoglycosidase is PNGase F.

In some embodiments, the suspension comprises 2, 3, 4, 5, 6, 7, 8, 9, or 10 distinct magnetic particles.

In some embodiments, the magnetic particle is a nanoparticle.

In some embodiments, the magnetic particle is a microparticle.

In some embodiment, the composition provided herein is used for detecting N-linked glycoproteins in the biological sample.

In some embodiments, detecting N-linked glycoproteins in the biological sample comprises performing the method provided herein.

In another aspect, provided herein is an apparatus comprising: (a) a plurality of partitions; (b) a plurality of reagent storages comprising an endoglycosidase and a protease, wherein the endoglycosidase and the protease are in the same or separate reagent storage; (c) one or more surfaces; (d) one or more transfer devices operably connected to the plurality of partitions, the plurality of reagent storages, and the plurality of substrates; and (e) a computer comprising at least one processor and instructions executable by the at least one processor to perform steps comprising: i. generating, using the one or more transfer devices, a suspension in one or more of the partitions, wherein the suspension comprises at least one of the one or more surfaces and a biological sample; ii. incubating the suspension to form a protein corona on the one or more surfaces; iii. isolating the protein corona from the suspension; and iv. digesting proteins from the protein corona using the endoglycosidase and protease, wherein the endoglycosidase and protease digest the proteins at the same or different times.

In some embodiments, the apparatus provided herein is used for detecting N-linked glycoproteins in the biological sample.

In some embodiments, detecting N-linked glycoproteins in the biological sample comprises performing the method provided herein.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Investigation of Plasma Proteome Using Incubation with Different Nanoparticles

Introducing a nanoparticle (NP) into a biofluid such as blood plasma leads to the formation of a selective, specific, and reproducible protein corona at the nano-bio interface driven by the relationship between protein-NP affinity, protein abundance, and protein-protein interactions. Since NP-coronas differentially interrogate complex samples at the proteoform level and plasma proteins are often glycosylated, here we investigate whether an automated NP-corona based proteomics workflow can interrogate the plasma glycoproteome without subsequent enrichment of glycosylated peptides. Protein glycosylation states can provide diagnostic evidence where total protein abundance is uninformative. Improved methods for profiling the plasma glycoproteome, especially at the lower abundance level thus can have a major impact in biomarker discovery.

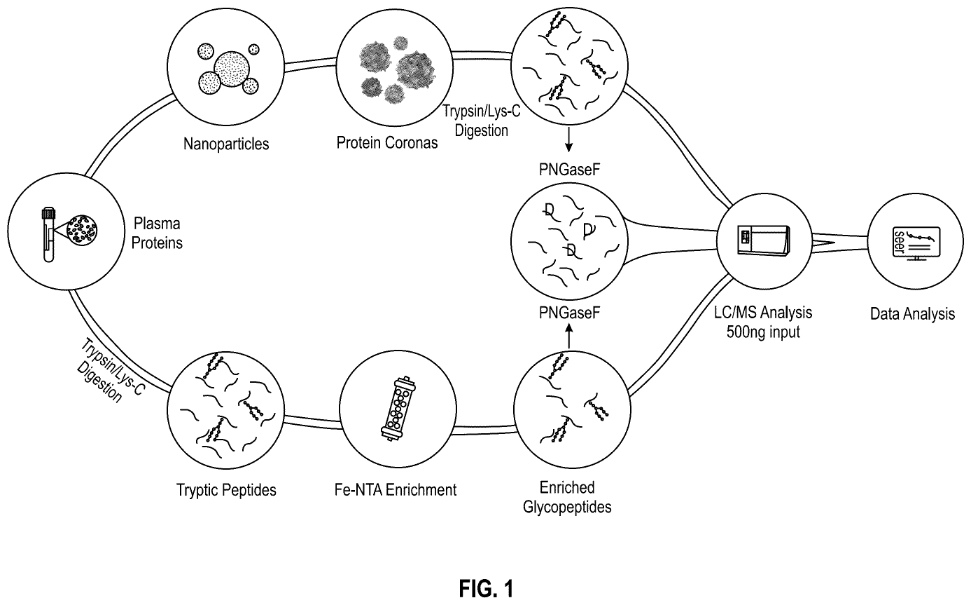

We have investigated the utility of NPs in capturing glycoproteins in comparison to neat plasma and a conventional glyco-enrichment workflow using magnetic Fe-NTA (nitrilotriacetic acid) beads. Samples were analyzed with Orbitrap Lumos mass spectrometer and UltiMate3000 Dionex LC system using 60 min DDA sample-to-sample runs. We evaluated depth, dynamic range, coverage, and precision of quantification at a wide range of concentrations for each NP. FIG. 1 shows a high-level summary of the workflow.

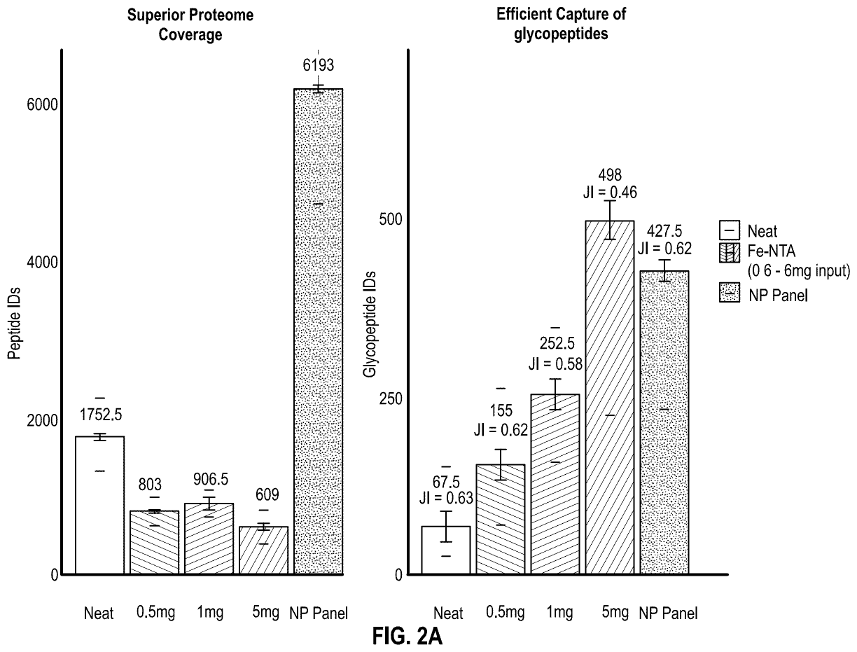

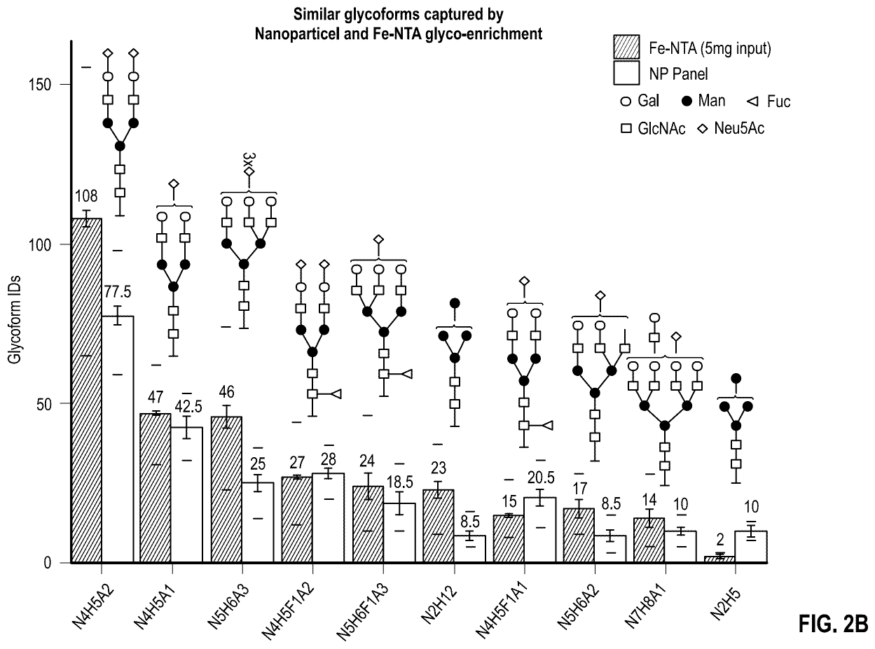

FIG. 2A shows the number of peptides and glycopeptides identified in a single pooled plasma: Neat, Fe-NTA enrichment using 0.5-5 mg of trypsin digested plasma, and NP panel (equivalent to 2 mg plasma protein per NP). These results did not use PNGaseF (peptide: N-glycosidase F) to cleave glycoproteins, but standard mass spectrometry techniques were used to identify glycoproteins. NP panel identifies more than 3.5× and 6× number of peptides compared to neat plasma and Fe-NTA enrichment, respectively. In addition, we identified a comparable number of glycopeptides in the NP panel and Fe-NTA enrichment with 5 mg plasma digest input. The glycopeptide identifications in the NP panel are more reproducible with JI (Jaccard Index) of 0.62 compared to 0.46 for the best Fe-NTA enrichment. The bar plots show the median number of IDs for three replicates. The top dash shows the number of features identified in any of the replicates and the bottom dash line shows the number of features commonly identified in all three replicates.

FIG. 2B shows the top 10 glycoforms identified in NP panel (142 overall) in comparison to the Fe-NTA enrichment with 5 mg input. Data show similar distribution of glycopeptides between the two methods. In addition, for each glycoform, the NP panel shows more reproducible identification (bottom dash) compared to Fe-NTA. Since in the NP panel we are not specifically enriching for glycopeptides, it enables glycopeptide identification with minimum bias toward any glycan structure. These results did not use PNGaseF to cleave glycoproteins, but standard mass spectrometry techniques were used to identify glycoproteins.

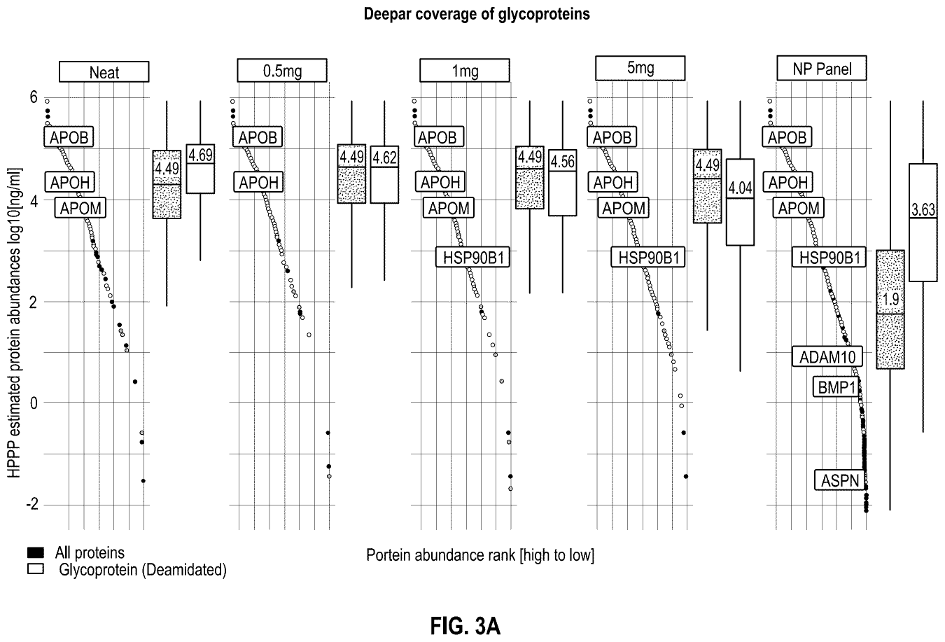

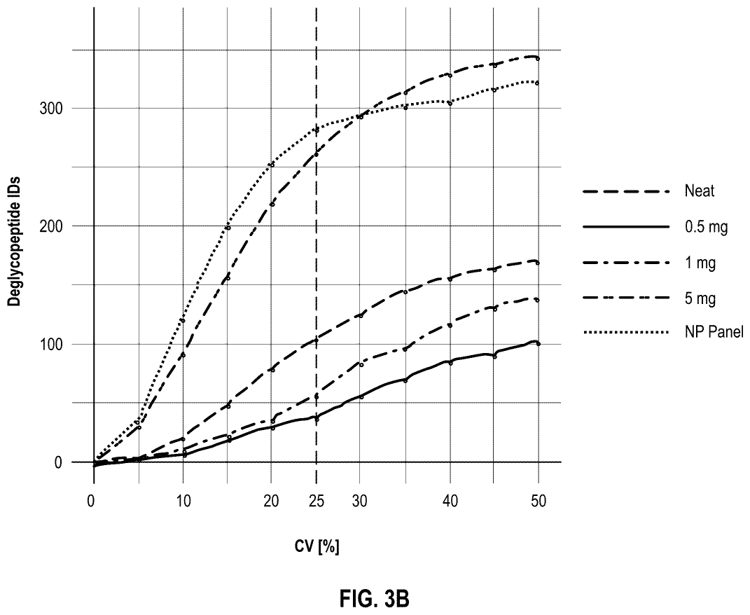

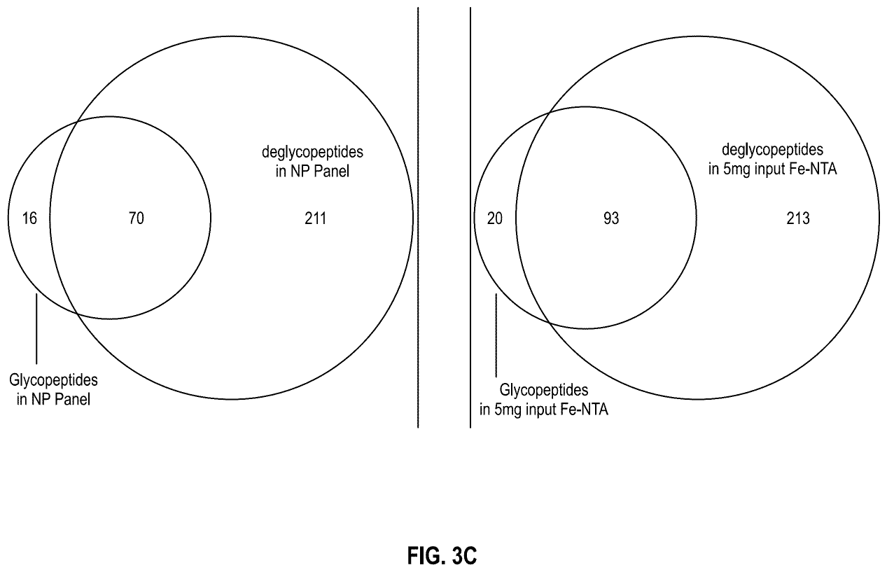

FIG. 3A shows depth of coverage in each workflow is shown based on the estimated protein abundance in the HPPP reference library. With the NP Panel, the median abundance of the identified peptides is more than 2 orders of magnitude lower than the other workflows. Consequently, using the NP Panel, we identify proteins with deglycopeptide evidence at the lower abundance range compared to neat plasma and the glyco-enriched workflows. In each workflow a few proteins that have at least one deglycopeptide evidence are highlighted. These proteins are known glycoproteins that have roles in lipid metabolism, neurodegeneration, heatshock and growth. These results did not use PNGaseF to cleave glycoproteins, but standard mass spectrometry techniques were used to identify glycoproteins.

FIG. 3B shows a CV accumulation plot for deglycopeptides. Using the NP Panel, we identify more deglycopeptides compared to the glyco-enriched workflow with high quantification reproducibility, and it is only at higher CVs that the number of deglycopeptide identifications in the glyco-enriched workflow surpasses the NP panel. These results did not use PNGaseF to cleave glycoproteins, but standard mass spectrometry techniques were used to identify glycoproteins.

FIG. 3C shows peptides from the NP Panel and Fe-NTA workflows that were subjected to PNGaseF treatment. The results suggest there are many glycopeptides that are not being identified prior to PNGaseF treatment.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The expressions “at least one of A and B” and “at least one of A or B” may be construed to mean at least A, at least B, or at least A and B (i.e., a set comprising A and B, which set may include one or more additional elements). The term “A and/or B” may be construed to mean only A, only B, or both A and B.

The expressions “at least about A, B, and C” and “at least about A, B, or C” may be construed to mean at least about A, at least about B, or at least about C. The expressions “at most about A, B, and C” and “at most about A, B, or C” may be construed to mean at most about A, at most about B, or at most about C.

The expression “between about A and B, C and D, and E and F” may be construed to mean between about A and about B, between about C and about D, and between about E and about F. The expression “between about A and B, C and D, or E and F” may be construed to mean between about A and about B, between about C and about D, or between about E and about F.

The expression “about A to B and C to D” may be construed to mean between about A and about B and between about C and about D. The expression “about A to B or C to D” may be construed to mean between about A and about B or between about C and about D.

The term “exemplary” as used herein means “serving as an example, instance, or illustration.” Any embodiment described herein as “exemplary” is not to be construed as preferred or advantageous over other embodiments.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising:
(a) incubating a composition comprising a plurality of different proteins with one or more surfaces to form a protein corona on the one or more surfaces;
(b) selectively cleaving N-linked saccharides on proteins from the protein corona; and
(c) assaying proteins from the protein corona to identify one or more N-linked glycoproteins in said composition.

2. The method of claim 1, wherein the composition has a pH between about 5 and 7.

3. The method of claim 1, wherein the composition has a pH between about 9 and 10.

4. The method of claim 1, wherein the composition comprises buffer.

5. The method of claim 4, wherein the composition comprises Tris buffer.

6. The method of claim 1, wherein the composition comprises at least about 1000, 100, 10, 1, 0.1, 0.01, or 0.001 nanograms of proteins.

7. The method of claim 1, wherein the composition comprises a complex biological sample, wherein the complex biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

8. The method of claim 1, wherein the one or more surfaces comprise a particle, wherein the particle comprises a porous particle, a microparticle, a nanoparticle, or any combination thereof.

9. The method of claim 8, wherein the particle comprises a paramagnetic material, wherein the paramagnetic material comprises superparamagnetic material, iron oxide, or any combination thereof.

10. The method of claim 1, wherein said composition is incubated with a first surface and a second surface having different physicochemical properties such that different protein coronas are formed on each surface.

11. The method of claim 10, wherein the first surface and the second surface comprise the same sign of charge, the same sign of zeta potential, or any combination thereof.

12. The method of claim 10, wherein the first surface, the second surface, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

13. The method of claim 1, wherein the assaying comprises mass spectrometry, wherein the mass spectrometry comprises LC-MS/MS.

14. The method of claim 1, wherein the assaying comprises affinity-based detection, liquid chromatography, protein sequencing, or any combination thereof.

15. The method of claim 1, wherein (b) comprises incubating proteins from the protein corona, or peptide fragments thereof, with an endoglycosidase;
wherein the endoglycosidase is PNGase F, PNGase A, Endoglycosidase H, Endoglycosidase F, α2-3,6,8,9 Neuraminidase A, β1-4 Galactosidase S, β-N-acetylhexosaminidase, or any combination thereof;
wherein a protease is incubated with proteins from the protein corona, at least in part, before (c), during (b), before (b), or any combination thereof;
wherein the protease is trypsin, Chymotrypsin, lysc, proteinase K, elastase, AspN, GluC, or combinations thereof;
wherein, at least in part during (b), an endoglycosidase and a protease are incubated with the proteins from the protein corona at the same time.

16. The method of claim 1, further comprising identifying a biological state associated with the composition based at least partially on the one or more N-linked glycoproteins detected in the composition;
wherein selectively cleaving N-linked saccharides on proteins from the protein corona comprises hydrolyzing an amide linkage;
wherein identifying one or more N-linked glycoproteins in said composition comprises detecting a protein having a deamidated residue, an aspartic acid residue substituted for an asparagine residue, or any combination thereof.

17. The method of claim 16, further comprising quantifying the one or more N-linked glycoproteins;
wherein N-linked glycoproteins are detected from a biological sample across a dynamic range of at least 6, 7, 8, 9, or 10 orders of magnitude;
wherein N-linked oligosaccharides are cleaved from proteins from the protein corona;
wherein the oligosaccharides include at least 3, 4, 5, 6, 7, 8, 9, 10, 15, 20, 25, or 50 monosaccharide units;
wherein at least 50, 100, 200, or 250 glycoproteins are detected in a biological sample.

18. A kit comprising:
(a) a protease;
(b) an endoglycosidase;
(c) a first dried composition comprising a first magnetic particle and a first stabilizer, wherein the first magnetic particle has a positive zeta potential; and
(d) a second dried composition comprising a second magnetic particle and a second stabilizer, wherein the second magnetic particle has a negative zeta potential.

19. A composition comprising:
a suspension comprising:
i. a magnetic particle having a protein corona formed on the surface of the magnetic particle, wherein the protein corona is obtained by incubating the magnetic particle with a biological sample; and
ii. an endoglycosidase.

20. An apparatus comprising:
(a) a plurality of partitions;
(b) a plurality of reagent storages comprising an endoglycosidase and a protease, wherein the endoglycosidase and the protease are in the same or separate reagent storage;
(c) one or more surfaces;
(d) one or more transfer devices operably connected to the plurality of partitions, the plurality of reagent storages, and the plurality of substrates; and
(e) a computer comprising at least one processor and instructions executable by the at least one processor to perform steps comprising:
i. generating, using the one or more transfer devices, a suspension in one or more of the partitions, wherein the suspension comprises at least one of the one or more surfaces and a biological sample;
ii. incubating the suspension to form a protein corona on the one or more surfaces;
iii. isolating the protein corona from the suspension; and
iv. digesting proteins from the protein corona using the endoglycosidase and protease, wherein the endoglycosidase and protease digest the proteins at the same or different times.
